(19) United States
(12) Patent Application Publication
(10) Pub. No.: US 2014/0012136 A1
(43) Pub. Date: Jan. 9, 2014

(54) Biological Optical Measuring Apparatus

(71) Applicant: Hitachi, Ltd., Tokyo (JP)

(72) Inventors: Daisuke SUZUKI, Tokyo (JP); Masashi KIGUCHI, Tokyo (JP)

(21) Appl. No.: 13/929,994

(22) Filed: Jun. 28, 2013

(30) Foreign Application Priority Data
Jul. 4, 2012 (JP) 2012-150067

(51) Int. Cl. A61B 5/00

(52) U.S. Cl. CPC A61B 5/721; A61B 5/0059
USPC 600/473

(57) ABSTRACT

A biological optical measuring apparatus includes a light source probe and a light receiving probe, one of which is provided with a pressure sensor to detect a contact pressure of a skin of a subject. Pairs of plural values of the contact pressure and light detection signals are previously recorded as calibration data, an estimated value of a false signal is derived from a detection value of the pressure sensor at primary measurement and the calibration data, and a measurement signal waveform in which a noise component due to a movement of the subject is removed is acquired by subtracting the estimated value from a light measurement signal.

### BACKGROUND OF THE INVENTION

1. Field of the Invention

The present invention relates to a biological measuring apparatus using optical measurement.

2. Background Art

A measuring apparatus called an optical topography apparatus is known as a biological optical measuring apparatus. This apparatus is such that many light source probes for light irradiation and many light-receiving probes for light reception are arranged on a biological measurement object, and a difference of transmitted light scattered in the biological body is measured, so that biological information, for example, a change of blood flow is measured.

The light source probes and the light-receiving probes are arranged on the skin of the measurement object while a predetermined inter-probe distance on the measurement object is secured. Since the surface of the biological body has concaves and convexes or curved surfaces, in order to absorb the concaves and convexes, the probe is constructed to come in contact with the skin while force is applied by a spring or the like. When the distribution of biological information is measured, many light source probes and many light-receiving probes are attached so as to come in close contact with a measurement part, for example, a head, each of the light source probes irradiates a near infrared ray, and each of the light receiving probes measures the scattered transmitted light.

In this optical topography apparatus, in order to measure the transmitted light, the contact state of the light source side probes and the light-receiving side probes to the biological body under measurement is required to be kept constant. If the contact state to the skin is changed, there is a fear that incident light intensity or received light intensity is changed irrespective of blood flow change of a tissue or the like, and a noise component (false signal) is superimposed on the measurement result. Thus, in general, when the optical topography measurement is performed, a subject is required not to move as much as possible, and the measurement is performed while the contact state of the probe is not changed. In order to handle the movement of the subject, JP-T-2005-535408 (Patent Literature 1) discloses a method in which an acceleration sensor is provided on a probe, the acceleration sensor measures the movement of the subject, and when movement larger than an allowable amount is detected, an assist signal indicating that a noise is superimposed on a measurement signal during the period is recorded. Besides, there is a method in which movement is measured by an acceleration signal, a noise amount is calculated based on the movement, and noise removal is performed. However, since the acceleration signal and the change of the contact state between the probe and the skin are significantly dependent on skin state, biological tissue state, and fixing state of the probe to the biological body, the acceleration and the noise amount are not necessarily correlated at high reproducibility.

When the subject is moved or the posture is changed during the measurement of the optical topography, a noise component (false signal) due to the change of the contact state of the probe and irrespective of the blood flow change is superimposed on the measurement signal, and the measurement of the blood flow change may not be accurately performed. Particularly, if the temporal response of change in brain blood flow and the temporal response of noise component due to the movement are in a similar frequency band, the separation of the signal from the noise is difficult. However, according a method of forcing the subject not to move or a method of invalidating measurement if a movement of generating a noise difficult to be separated from a signal occurs and again performing measurement, a burden imposed on the subject is high. Accordingly, the use of only these methods becomes a factor of inhibiting the widening of application range of this kind of biological measuring apparatus.

### SUMMARY OF THE INVENTION

Therefore, an object of the invention is to provide a biological optical measuring apparatus in which the occurrence of discarding of measurement results and remeasurement due to movement of a subject is reduced and a burden on the subject is low.

According to an aspect of the invention, a biological optical measuring apparatus includes at least one probe provided with a sensor capable of detecting a contact pressure between the probe and a skin, and before blood flow measurement, calibration measurement is previously performed to estimate a degree of a noise signal superimposed on a measurement signal when the contact pressure is changed. Since the calibration result of the pressure change and the superimposed noise signal (false signal) is significantly changed by a state of the skin of a subject and a state when the probe is mounted, the calibration is performed each time the probe is mounted on the subject. A method of the calibration measurement is such that the subject is relaxed so as not to increase a blood flow, a pressure is applied to the probe, and a pressure signal and optical topography signals at that time are measured. As a method of applying the pressure to the probe, a method of directly applying a pressure to each probe or a method in which the posture of the subject is inclined, and the direction of gravity received by the probe is changed to change the contact pressure is used. Based on the calibration measurement, even when the contact pressure is changed by movement or the like during primary measurement and a noise is superimposed, the superimposed signal is subtracted by using the simultaneously measured pressure signal, so that a target signal caused by a change in blood flow can be corrected and calculated.

In the related art optical topography measurement, if a measurement part is inclined or is moved during the measurement, a noise component (false signal) is superimposed, and the original target signal caused by a change in blood flow is buried in a noise component and sometimes can not be determined. According to the method of the invention, even if the movement of the subject or the like occurs during the measurement, the signal during the period can be effectively measured.

Besides, in measurement under a condition where a subject is moved, a method is used in which a burden asynchronous with the movement is periodically applied to the subject, and averaging of measurement results is performed to remove a noise component due to the movement. However, in that case, since the measurement is performed plural times, the measurement time becomes long. On the other hand, according to this method, the number of times of addition decreases, or the averaging is not required, and the measurement can be performed in a short time.

### BRIEF DESCRIPTION OF THE DRAWINGS

FIG. 1 is a perspective view showing the whole structure of a biological optical measuring apparatus of an embodiment.

FIG. 2 is a block circuit view of a probe and a measurement control circuit of the embodiment.

FIG. 3 is a sectional view showing a vertical section of a light source probe of the embodiment.

FIG. 4 is a sectional view showing a vertical section of a light receiving probe of the embodiment.

FIG. 5 is a flowchart of a measurement procedure of the embodiment.

FIG. 6 is a waveform view showing an example of noise removal of a topography signal by pressure data.

FIG. 7 is a sectional view showing a horizontal section of a light receiving probe provided with a three-axis pressure sensor.

FIG. 8 is a flowchart of a measurement procedure of a modified example in which a noise component is derived from a look-up table.

### DESCRIPTION OF EMBODIMENTS

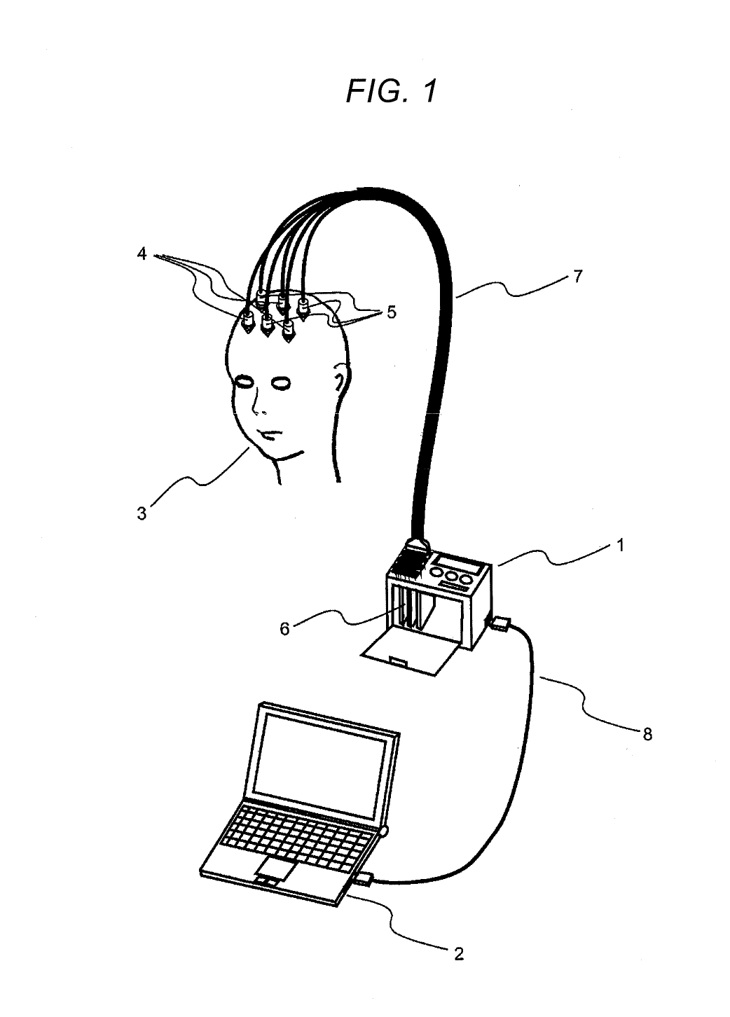

Hereinafter, a biological optical measuring apparatus of an embodiment will be specifically described with reference to FIG. 1 to FIG. 8. The optical measuring apparatus of this embodiment is such that when a certain part of a brain is activated, an amount of blood for feeding oxygen to the part is increased accordingly, and this is used to measure a local blood dynamic change in a biological body. Specifically, a near infrared ray is irradiated from above to a head skin, and scattering of the near infrared ray by hemoglobin in blood is measured, so that a change in blood amount near a cerebral surface is measured. This is expressed in a two-dimensional map or the like, and the brain activity can be easily observed. Here, the near infrared ray is an electromagnetic wave in a wavelength region longer than visible light.

FIG. 1 is a perspective view showing the whole measurement system. An optical measuring apparatus measures a change in a blood amount of a measurement object 3. A main body 1 of the optical measuring apparatus includes plural measurement control circuits 6. Plural light source probes 4 and plural light receiving probes 5 are connected to the main body 1 of the measurement control circuits through a signal cable 7. Further, a data recording control device 2 is connected to the main body 1 through a control line cable 8, and the measurement system is constructed as described above.

In order to measure the blood change of the measurement object, each of the probes is placed in contact with the skin of the measurement object and the measurement is performed. The measurement control circuits 6 perform the control of light source intensity and light emitting timing, numerical conversion in a light receiving sensor, digitization in a pressure sensor for measuring a contact pressure between the probe and the skin, and the like. The optical measuring apparatus is controlled by the data recording control device connected to the measurement control circuits. The connection between the data recording control device and the measurement control circuits may be performed by a wireless system instead of by the control line cable 8.

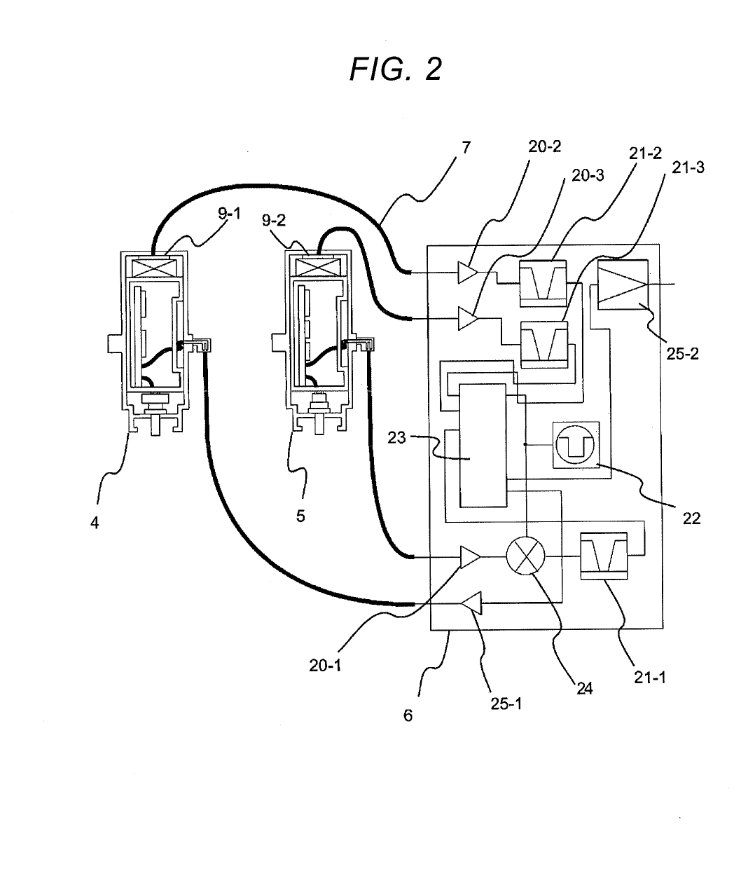

FIG. 2 is a view showing the optical measuring apparatus for one channel. The light source probe 4 and the light receiving probe 5 are paired for one measurement area and measurement is performed. At least one of the pair of probes is provided with a pressure sensor, and the contact pressure between the probe and the skin during measurement can be measured. In the illustrated example, the light source probe is provided with a pressure sensor 9-1, and the light receiving probe 5 is also provided with a pressure sensor 9-2. When an in-plane distribution of a wide area is measured, a structure can be adopted in which plural pairs of probes are arranged and the measurement is performed. In this case, the structure may be such that one probe is provided with a pressure sensor, both probes of one pair are provided with pressure sensors, or all probes are provided with pressure sensors.

Each of the probes is controlled by the measurement control circuit 6. A modulated light control signal generated by a microcomputer 23 is outputted to the light source probe 4 through a buffer 25-1. A light detection signal of the light receiving probe 5 is transmitted to the microcomputer 23 through a signal amplifier 20-1, a synchronous detector 24 and a band-pass filter 21-1. The synchronous detector 24 synchronously detects the light detection signal based on a reference signal outputted by a clock 22. Pressure detection signals from the pressure sensors 9-1 and 9-2 are respectively transmitted to the microcomputer 23 through a signal amplifier 20-2 and a filter 21-2, and a signal amplifier 20-3 and a filter 21-3. Besides, the microcomputer 23 digitizes and captures the light detection signal and the pressure detection signal, and transmits them to the data recording control device 2 through a buffer 25-2.

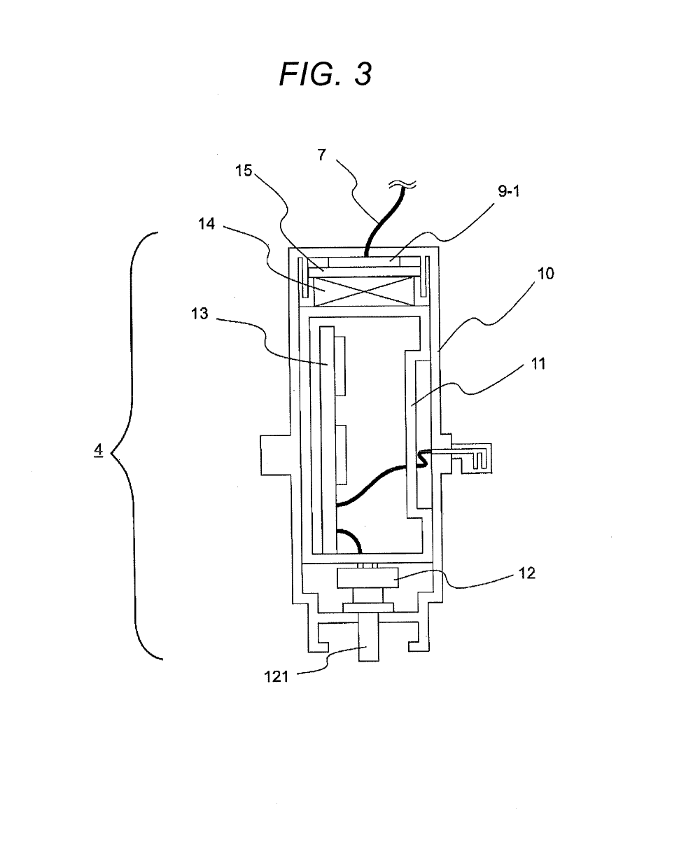

FIG. 3 is a vertical sectional view of the light source probe 4. The light source probe 4 includes a probe case 10, a working part 11, a light source 12, an optical guide 121, a light source driving circuit 13, a spring 14, a pressure sensor 9-1, a press plate 15 and a signal cable 7. The working part 11 is pressed by the spring 14. Accordingly, the light source 12 and the light guide 121 having an end protruding from the probe case 4 are also pressed by the repulsive force of the spring 14. By this structure, when the probe case 14 is mounted on the measurement object 3 by a mounting member not shown in the drawing, the concaves and convexes of the measurement object are absorbed and the end of the optical guide 121 can be placed in press contact with the skin of the measurement object 3 within a specified pressure range. The pressure sensor 9-1 is arranged between the probe case 10 and the press plate 15. When the spring 14 is pressed by the working part 11, the pressure at that time, that is, the contact pressure between the optical guide 121 and the measurement object can be measured. The press plate 15 is arranged so that the pressure of the working part 11 and the probe case 10 is uniformly applied.

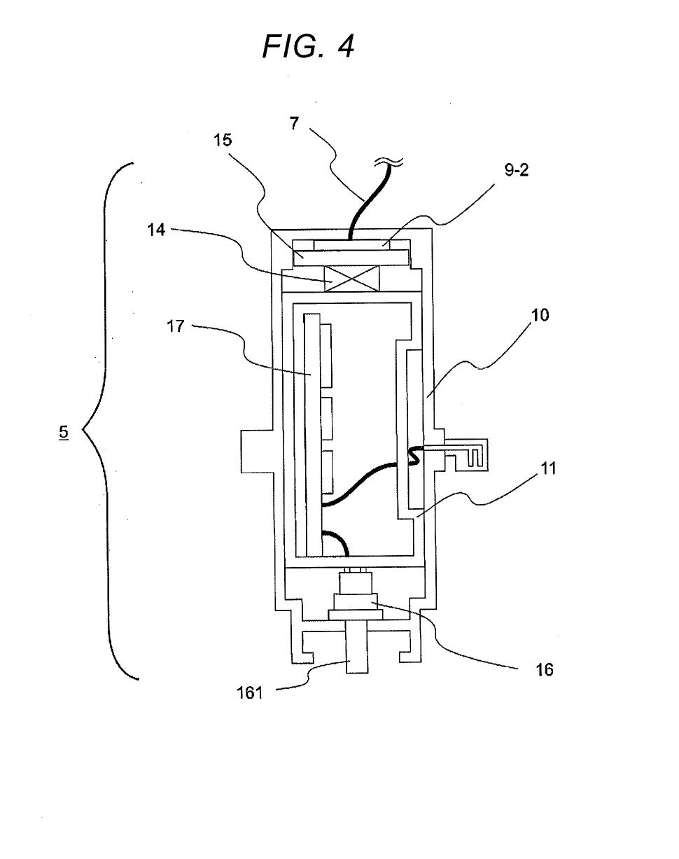

FIG. 4 is a vertical sectional view of the light receiving probe 5. The light receiving probe 5 includes a probe case 10, a working part 11, a light receiving sensor 16, an optical guide 161, a light receiving sensor circuit 17, a spring 14, a pressure sensor 9-2, a press plate 15 and a signal cable 7. Portions having the same structures and same functions as the portions of the light source probe 4 are denoted by the same reference numerals. That is, in the light receiving probe, the working part 11, the light receiving sensor 16 and the optical guide 161 are pressed by the spring 14. Similarly to the light source probe, the concaves and convexes of the head of the measurement object are absorbed, and the optical guide 161 is placed in press contact with the head within a specified pressure range. Besides, the contact pressure between the optical guide 161 and the measurement object is measured by the pressure sensor.

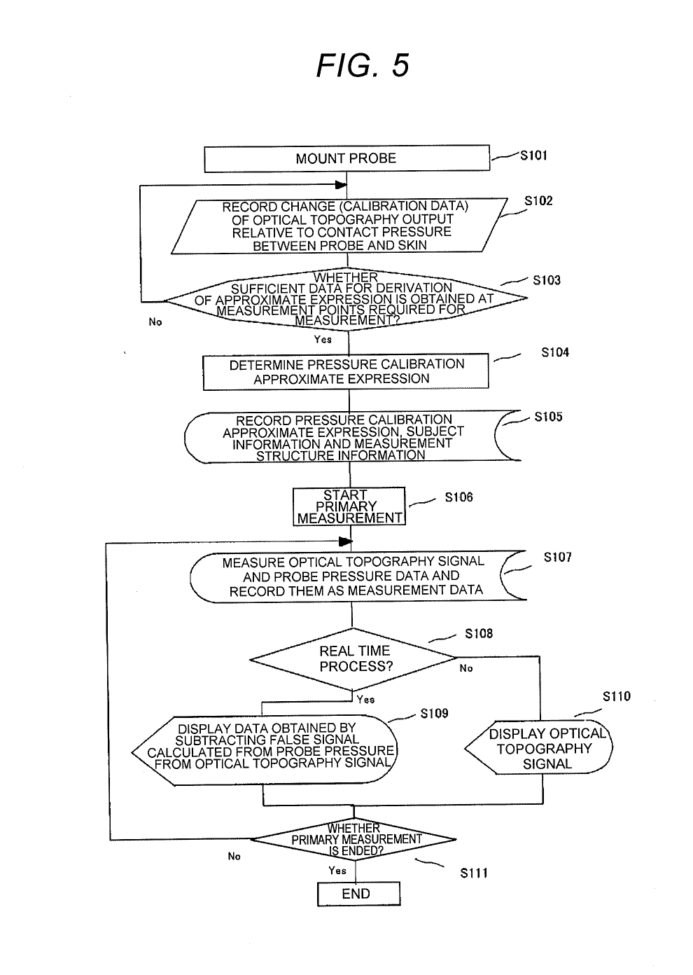

FIG. 5 is a flowchart showing a procedure of calibration measurement and primary measurement of the embodiment. First, at S101, the probes are mounted on the subject, and the measurement preparation is performed. Next, at S102, the calibration measurement, that is, calibration data is recorded. At this time, force is applied to each of the probes, and the calibration data of the relevant channel is measured. The force applied to the probe is sequentially changed so as to include the range of the probe contact pressure (pressure of contact between the end of the optical guide of the probe and the skin) changed by the movement of the subject and the like at the time of the primary measurement, and the optical topography output corresponding to each contact pressure is recorded as the calibration data. During the calibration measurement, the measurement is performed while the subject is relaxed so as not to cause blood flow change. The optical topography output obtained in this way does not reflect the brain activity of the subject, but is a signal entirely dependent on the contact pressure of the probe, and can be regarded as a false signal mixed in the measurement of the brain blood flow signal. The calibration data recording is repeated while the contact pressure is changed until it is determined at S103 that sufficient data for derivation of an approximate expression used for calibration at measurement points required for the measurement is obtained. Next, at S104, the recorded calibration data (pair of pressure and false signal) is used, and a function (pressure calibration approximate expression) is determined in which when an input variable is the pressure, an output is the false signal.

The function obtained here is a first- to fifth-degree polynomial function. Typically, the second-degree polynomial function can suitably approximate the false signal corresponding to the pressure. In this case, the process at S104 is the process of obtaining coefficients A, B and C of the expression (numerical expression 1) by using the recorded calibration data.

T=A+Bx+Cx² (1)

Where, x denotes a pressure value, and T denotes a topography signal value (false signal).

Next, at S105, the determined pressure calibration approximate expression, together with subject information, measurement structure information and the like, is recorded. The recording of the calibration data, the derivation of the approximate expression, and recording are performed by the data recording control device 2.

The primary measurement is performed after S106. In the measurement of brain blood dynamic change, in general, a stimulus is given to the subject or a burden is applied to the subject, and a local state change of the brain thereto is observed through the waveform obtained from the optical topography signal. The primary measurement here is often the measurement including the giving of the stimulus or the execution of the problem. At S107, data of the optical topography signal of the primary measurement and data of probe contact pressure during the measurement are acquired and recorded. In the primary measurement, there is a method of removing the noise component approximated by the pressure data from the measurement data at any time during the measurement, or a method of recording the measurement data and the pressure data and removing the noise component from the measurement data by using the approximate expression after the measurement is ended. At S108, it is determined whether the process mode set in the data recording control device 2 is the process (real time process) in accordance with the former method. If the determination indicates the real time process, at 109, the data recording control device 2 substitutes the pressure detection value into the predetermined pressure calibration approximate expression to estimate the value of the false signal, and subtracts the false signal estimated value from the light detection signal value obtained by the measurement. As a result, the data of the optical topography signal in which the noise component is removed is obtained. Besides, the response waveform indicated by the data is displayed on the data recording control device 2. If the determination at S108 does not indicate the real time process, the response waveform indicated by the transmitted light detection signal value is directly displayed at S110. Incidentally, when the method of removing the noise component at any time is adopted, there is a method of causing the approximate expression to be reflected on the measurement control circuit and recording the data in which the noise is removed, or a method of causing the data recording control device to remove the noise component derived by using the approximate expression and to record.

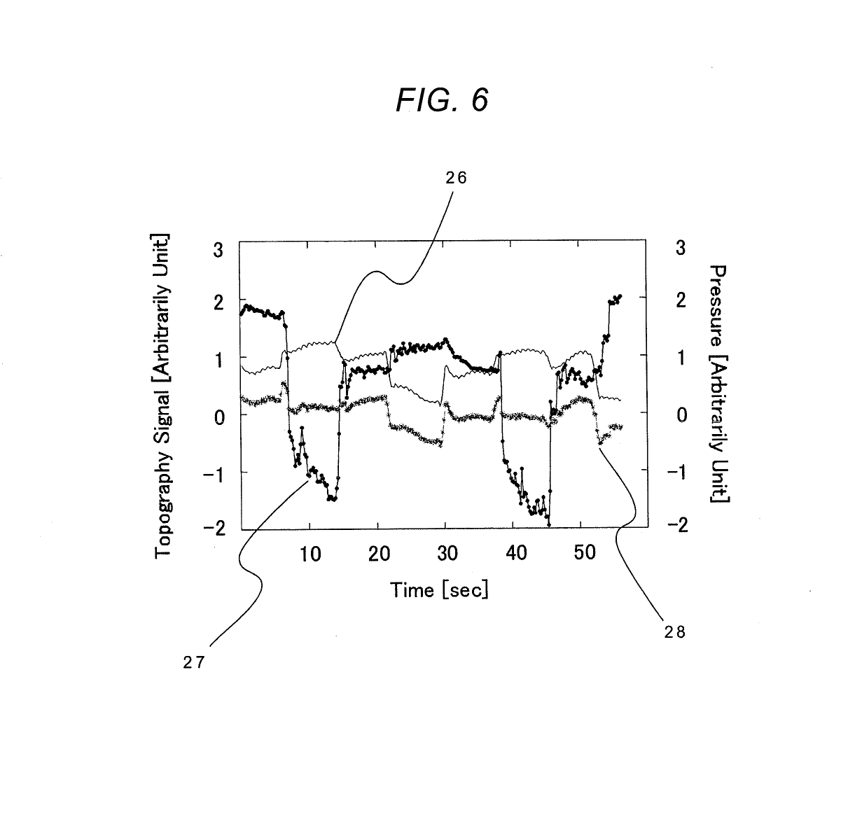

FIG. 6 is a view showing an example in which a second-degree polynomial function is calculated as a pressure calibration approximate expression based on the pressure signal of the light receiving probe actually obtained from the pressure sensor, and the noise component is removed from the topography signal. In the drawing, the horizontal axis indicates the time, and the vertical axis indicates the topography signal intensity and the probe pressure value. The units of both are arbitrary. A solid line 26 indicates the topography signal before the noise component is removed. A circle and solid line 27 indicates the probe contact pressure value. A star and solid line 28 indicates the topography signal in which the false signal (noise component) converted from the pressure is removed. In this example, the polynomial function of the numerical expression 1 was used as the pressure calibration approximate expression. The values of the coefficients derived by using the recorded calibration data were A=0.93420, B=−0.181892 and C=−0.023618.

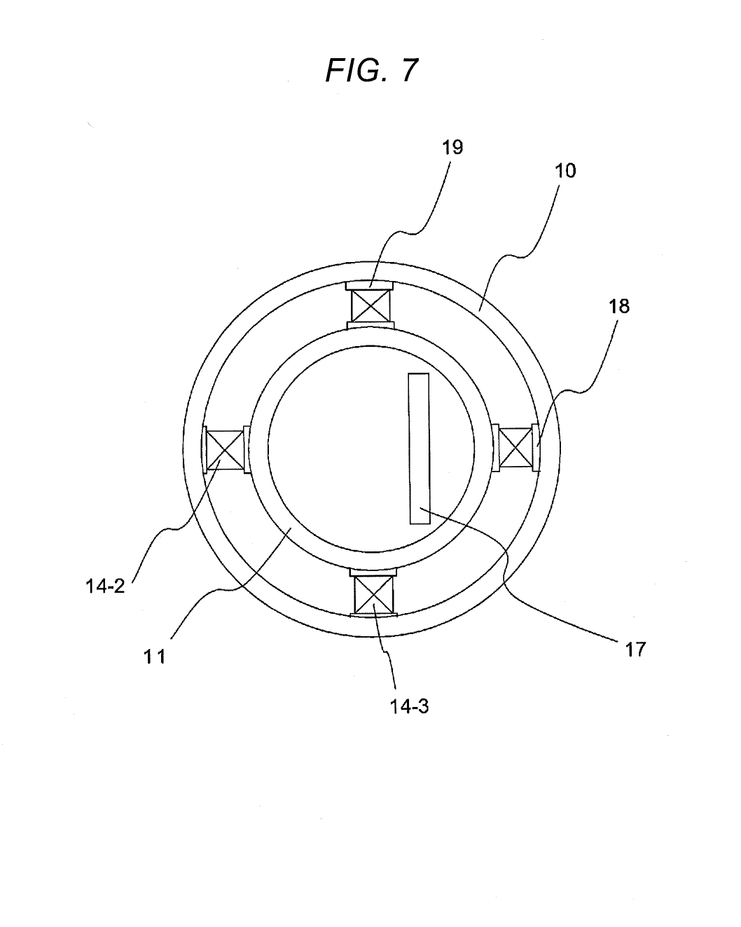

In the embodiment described above, the pressure sensor provided in the light source probe or the light receiving probe detects the contact pressure in the perpendicular direction to the skin of the subject. However, the change of the pressure caused by the movement of the subject applied to the probe mounted so as to be pressed to the subject includes not only the pressure change in the perpendicular direction but also the pressure change in the lateral direction. The light detection signal of the probe is influenced also by the pressure change in the lateral direction. Then, modification is effective in which a two-axis pressure detector in the lateral direction is provided in the probe in addition to the pressure detector in the perpendicular direction, and the pressures in the three axes in total are detected. FIG. 7 shows a structure of a light receiving probe used in a modified example in which pressures in the three axes are detected to store calibration data, the false signal is estimated by the pressures in the three-axis directions, and the calibration of the topography signal is performed. FIG. 7 is a sectional view showing a horizontal section vertical to the axis of the probe. In a probe case 10, a working part 11 pressed by a not-shown spring (see FIG. 4) in the vertical direction is sandwiched between a spring 14-2 and an x-direction pressure sensor 18 and between a spring 14-3 and a y-direction pressure sensor 19, and is disposed in the probe case. Pressure in the lateral direction applied to an end of a light guide similar to that shown in FIG. 4 is detected by the x-direction pressure sensor 18 and the y-direction pressure sensor 19. If coefficients of an extended polynomial function of the foregoing numerical expression 1, that is, coefficients of a polynomial function with variables of the vertical direction contact pressure, the x-direction pressure and the y-direction pressure are specified based on the result of the calibration measurement, the polynomial function to estimate the false signal by the pressures in the three-axis directions is obtained.

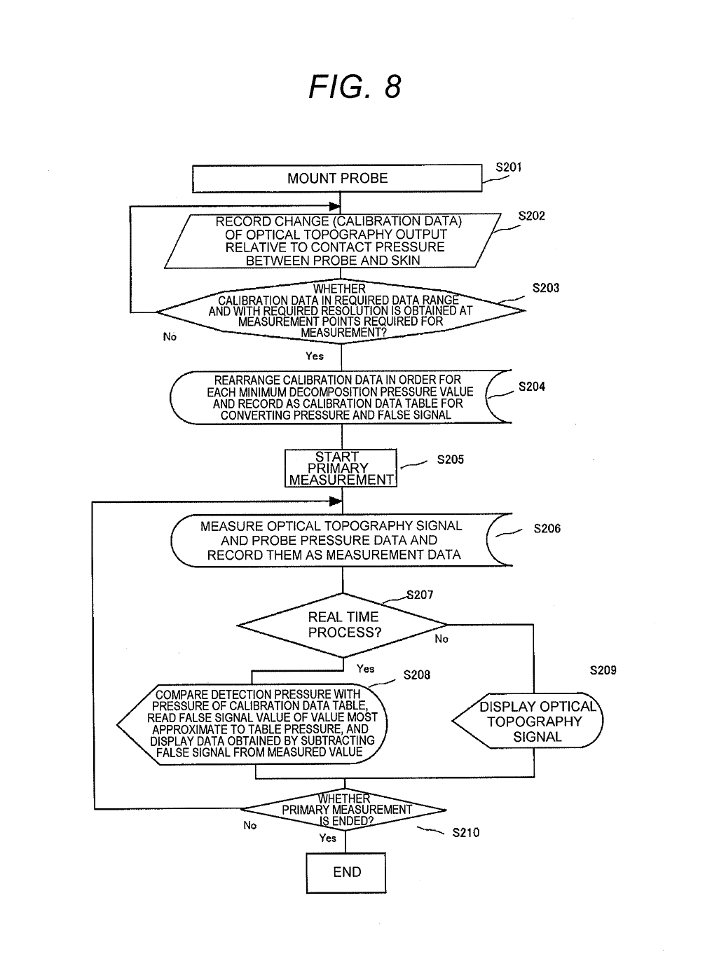

FIG. 8 is a flowchart showing a modified example of the procedure of the calibration measurement and the primary measurement. In this example, the procedure of measurement preparation and until calibration data recording by the calibration measurement at S201 to S203 are the same as those at S101 to S103 of FIG. 5. Next, at S204, instead of deriving the pressure calibration approximate expression, probe contact pressures in the calibration measurement and detection values of optical topography output are recorded in a table. Specifically, the calibration data are rearranged in order for each minimum decomposition pressure value, and are recorded as a conversion table of pressures and false signals. At this time, force is applied to each probe, and calibration data of the relevant channel is measured. During the measurement, the applied force is changed within the range in which a force corresponding to a pressure changed by movement or the like is sufficiently contained, and the calibration data is recorded. At this time, there is a method of applying the force to each of the probes or a method of changing the posture of a subject in many directions. The calibration measurement is performed while the subject is relaxed so as not to cause blood flow change. In FIG. 7, the calibration data table corresponding to the pressure sensors is recorded, and the procedure of the primary measurement indicated at S203 to S210 is basically the same as the procedure of S104 to S111 of FIG. 5. However, at S208 of the stage of removing a noise component from a light detection signal, a specific method is different. That is, at S208, the detection value of the probe contact pressure in the primary measurement is compared with the pressure recorded in the calibration data table, and among the pressures recorded in the calibration data table, a value of a false signal corresponding to the pressure closest to the detection value of the contact pressure is read, and the calibration is performed by subtracting the read value of the false signal from the value of the light detection signal. In this way, in the procedure of FIG. 8, instead of that the probe contact pressure is substituted into the pressure calibration approximate expression to estimate the noise component and the calibration is performed, the noise component is estimated by the look-up table system. In order to obtain the waveform of the blood movement suitably expressing the brain activity, it is required that the calibration measurement is performed in a smaller change width of contact pressure as compared with the procedure of FIG. 5, and the calibration data table is prepared.

According to the invention, the noise component mixed in the waveform of optical topography measurement can be effectively removed, the allowance for the movement of a subject in the measurement is increased, and the burden of the subject can be reduced. Accordingly, it is expected that the application of this type of apparatus is promoted.

What is claimed is:

1. A biological optical measuring apparatus comprising:
a light source probe which is applied to a measurement object and includes a mechanism to bring a first light guide as a passage of an irradiation light from a light source into press contact with a skin of the measurement object;
a light receiving probe which is applied to the measurement object and includes a mechanism to bring a second light guide as a passage of a light from the measurement object to the light receiving sensor into press contact with the skin of the measurement object;
a measurement control circuit which controls driving of the light source and captures a light detection signal from the light receiving probe, in which inner information of the measurement object is measured from an intensity change of the light scattered and transmitted through an inside of the measurement object;
a pressure sensor which is provided in at least one of the light source probe and the light receiving probe and detects a contact pressure between the measurement object and one of the first light guide and the second light guide; and
a data recording control device which correlates values of light detection signals of the light receiving sensor with a plurality of values of the contact pressures obtained from the pressure sensor at a time of previous calibration measurement, records pairs of those values as calibration data, and obtains data, which indicates a dynamic change of the inside of the measurement object and in which a noise component is removed, by subtracting an estimated value of a false signal based on the calibration data from the detection signal value of the light receiving sensor at a time of primary measurement.

2. The biological optical measuring apparatus according to claim 1, wherein the data recording control device determines a pressure calibration approximate expression to convert the contact pressure into the false signal based on the calibration data, and subtracts the estimated value of the false signal obtained by substituting a detection value of the contact pressure at a detection time point of the detection value of the light measurement signal into the pressure calibration approximate expression from the detection value of the light measurement signal at the time of the primary measurement.

3. The biological optical measuring apparatus according to claim 2, wherein the pressure calibration approximate expression is a polynomial function with the contact pressure as a variable, coefficients of the polynomial function are determined based on the calibration data, and the pressure calibration approximate expression is specified.

4. The biological optical measuring apparatus according to claim 1, wherein the data recording control device stores pairs of the plurality of values of the contact pressures obtained from the pressure sensor at the time of the calibration measurement and the values of the light detection signals of the corresponding light receiving sensor in a calibration data table, and obtains the estimated value of the false signal by reading a value of the light detection signal corresponding to the contact pressure of a value closest to the contact pressure at the detection time point of the detection value of the light measurement signal among the values of the contact pressures recorded in the calibration data table.

5. The biological optical measuring apparatus according to claim 1, wherein one of the light source probe and the light receiving probe includes a horizontal pressure sensor to detect a pressure in a horizontal direction applied to the light guide, and the calibration data is acquired for various values of outputs of the pressure sensor and the horizontal pressure sensor.
